(19) United States
(12) Patent Application Publication
(10) Pub. No.: US 2006/0084850 A1
(43) Pub. Date: Apr. 20, 2006

(54) System, method and apparatus for evaluating military personnel

(75) Inventors: Steven Jonathan Spinner, Menlo Park, CA (US); Steven John Fleck, Woodland Park, CO (US)

Correspondence Address: Robert E. Krebs;Thelen Reid & Priest LLP, P. O. Box 640640, San Jose, CA, 95164-0640, US

(73) Assignee: Sports Potential Inc., a Delaware Corporation

(21) Appl. No.: 11/039,490

(22) Filed: Jan. 6, 2005

Related U.S. Application Data
(63) Continuation-in-part of application No. 10/622,057, filed on Jul. 16, 2003.

(51) Int. Cl. A61B 5/00; A63B 69/00

(52) U.S. Cl. 600/301; 128/923; 434/247

(57) ABSTRACT

A participant is evaluated to determine compatibility among different military disciplines. The participant is first subjected to physical, physiological, and psychological tests. The results of tests are compared with a database that contains the results of the same tests subjected to a set of elite military members from different military disciplines. At least one specific military discipline compatible with the participant based on the comparison is identified. A list of identified specific disciplines from most compatible to least compatible associated with a corresponding match score is then generated.

### CROSS-REFERENCE TO RELATED APPLICATIONS

The present application is a continuation-in-part of application Ser. No. 10/622,057, filed Jul. 16, 2003, entitled “System, Method, and Apparatus for evaluating a person's athletic ability”.

### FIELD OF THE INVENTION

The present invention relates to the field of physical activity including sports. More particularly, the present invention relates to a field of identification of physical activity more suitable for an individual.

### BACKGROUND OF THE INVENTION

The problem of obesity among adults and more pertinently among children in America has been widely publicized. In California alone, it is estimated that almost 7 million (77 percent) youths physically unfit, and nearly 3 million (30 percent) are overweight. A CDC study showed that nearly half of American youths aged 12 to 21 years are not vigorously active on a regular basis. The CDC has identified two of the factors preventing youths from participating in regular physical activity: (1) a lack of opportunity to engage in enjoyable activities that promote the youths' confidence in their abilities, and (2) a limitation on the range of extracurricular programs that meet the needs and interests of specific populations, such as racial minorities, females and low-income groups.

These factors may be attributed to the lack of information on and access to a wide range of sporting activities. In many cases, children are introduced only to one or two sports during their formative years. Typically these are the sports that are prevalent within their geographic region and among their socioeconomic peers and may be sports for which they, as individuals are not well suited. Too often performance based criteria eliminates young people's participation in high school sports long before they graduate leading to a negative experience with sporting activities and a diminished self-confidence in their athletic abilities. At best, the individual will take the considerable initiative to seek out other sports, but more likely, the individual will eventually discontinue participation in sports all together.

It is also true that our society is becoming more attuned to the need for lifelong sporting and physical fitness activity to maintain health throughout ones life and independent living as one ages. Parents are motivated to create positive experiences for their children and to instill health-promoting habits at an early age. An increasing number of older adults and seniors are becoming aware of the benefits of physical activity and are beginning to seek out activities suitable to their physical capabilities. In academic and career domains, evaluation and guidance are commonly accepted practices. Children are tested regularly on their academic performance and aptitude in order to direct their academic pursuits; youths and adults undergo career skills assessments to provide career guidance and performance evaluations; the military uses sophisticated leadership performance evaluations to help in making informed decisions.

But in sports, individuals are largely left to fend for themselves in understanding their natural athletic talents, as well as in realistically assessing their progress in athletic pursuits. If any assessment is available, it is likely to be based on a coach's subjective opinion without reference to a suitable comparison group. This approach can prove highly unreliable and is not predictive. It will most likely be based on the coach's sports-specific experience and a very limited evaluation of the individual's physical capabilities. If an individual is fortunate enough to gain the attention of a sports organization, they may receive more advice on their capabilities, but only for that particular sport.

Therefore, a need exists for a system based on scientific and objective data that identifies which sports an individual is best suited. The system should enable individuals of all ages to make informed decisions about which sports to participate in based on insights into their physical and mental capabilities, as well as their personal preferences and interests. A primary purpose of the present invention is to solve these needs and provide further, related advantages.

### BRIEF DESCRIPTION OF THE INVENTION

A participant is evaluated to determine compatibility among different military disciplines. The participant is first subjected to physical, physiological, and psychological tests. The results of tests are compared with a database that contains the results of the same tests subjected to a set of elite military members from different military disciplines. At least one specific military discipline compatible with the participant based on the comparison is identified. A list of identified specific disciplines from most compatible to least compatible associated with a corresponding match score is then generated.

### BRIEF DESCRIPTION OF THE DRAWINGS

The accompanying drawings, which are incorporated into and constitute a part of this specification, illustrate one or more embodiments of the present invention and, together with the detailed description, serve to explain the principles and implementations of the invention.

In the drawings:

FIG. 1 is a diagram schematically illustrating a system for evaluating a person's athletic ability in accordance with one embodiment of the present invention.

FIG. 2 is a diagram schematically illustrating a system for evaluating a person's athletic ability in accordance with another embodiment of the present invention.

FIG. 3 is a diagram schematically illustrating a computer system suitable for implementing aspects of the present invention.

FIG. 4 is a flow diagram illustrating a method for evaluating a person's athletic ability in accordance with one embodiment of the present invention.

FIG. 5 is a table illustrating an example of test results on a set of athletes in four different physical activities with the test results of a subject in accordance with one embodiment of the present invention.

FIG. 6 is a graph illustrating an example of test results on a set of sprinters, long distance runners, weightlifters, basketball centers, in accordance with one embodiment of the present invention.

FIG. 7 is a graph illustrating an example of identifying a physical activity more compatible with a test subject in accordance with one embodiment of the present invention.

FIG. 8 is a flow diagram illustrating a method for evaluating military personnel in accordance with another embodiment of the present invention.

FIG. 9 is a graph illustrating an example of test results on a set of Army Rangers, Navy Seals, Green Berets, and Army Delta Force.

### DETAILED DESCRIPTION

Embodiments of the present invention are described herein in the context of a system, method, and apparatus for evaluating a person's athletic ability. Those of ordinary skill in the art will realize that the following detailed description of the present invention is illustrative only and is not intended to be in any way limiting. Other embodiments of the present invention will readily suggest themselves to such skilled persons having the benefit of this disclosure. Reference will now be made in detail to implementations of the present invention as illustrated in the accompanying drawings. The same reference indicators will be used throughout the drawings and the following detailed description to refer to the same or like parts.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application- and business-related constraints, and that these specific goals will vary from one implementation to another and from one developer to another. Moreover, it will be appreciated that such a development effort might be complex and time-consuming, but would nevertheless be a routine undertaking of engineering for those of ordinary skill in the art having the benefit of this disclosure.

In accordance with one embodiment of the present invention, the components, process steps, and/or data structures may be implemented using various types of operating systems (OS), computing platforms, firmware, computer programs, computer languages, and/or general-purpose machines. The method can be run as a programmed process running on processing circuitry. The processing circuitry can take the form of numerous combinations of processors and operating systems, or a stand-alone device. The process can be implemented as instructions executed by such hardware, hardware alone, or any combination thereof. The software may be stored on a program storage device readable by a machine.

In addition, those of ordinary skill in the art will recognize that devices of a less general purpose nature, such as hardwired devices, field programmable logic devices (FPLDs), including field programmable gate arrays (FPGAs) and complex programmable logic devices (CPLDs), application specific integrated circuits (ASICs), or the like, may also be used without departing from the scope and spirit of the inventive concepts disclosed herein.

In accordance with one embodiment of the present invention, the method may be implemented on a data processing computer such as a personal computer, workstation computer, mainframe computer, or high performance server running an OS such as Solaris® available from Sun Microsystems, Inc. of Palo Alto, Calif., Microsoft® Windows® XP and Windows® 2000, available form Microsoft Corporation of Redmond, Wash., or various versions of the Unix operating system such as Linux available from a number of vendors and other sytems not yet available. The method may also be implemented on a multiple-processor system, or in a computing environment including various peripherals such as input devices, output devices, displays, pointing devices, memories, storage devices, media interfaces for transferring data to and from the processor(s), and the like. In addition, such a computer system or computing environment may be networked locally, or over the Internet.

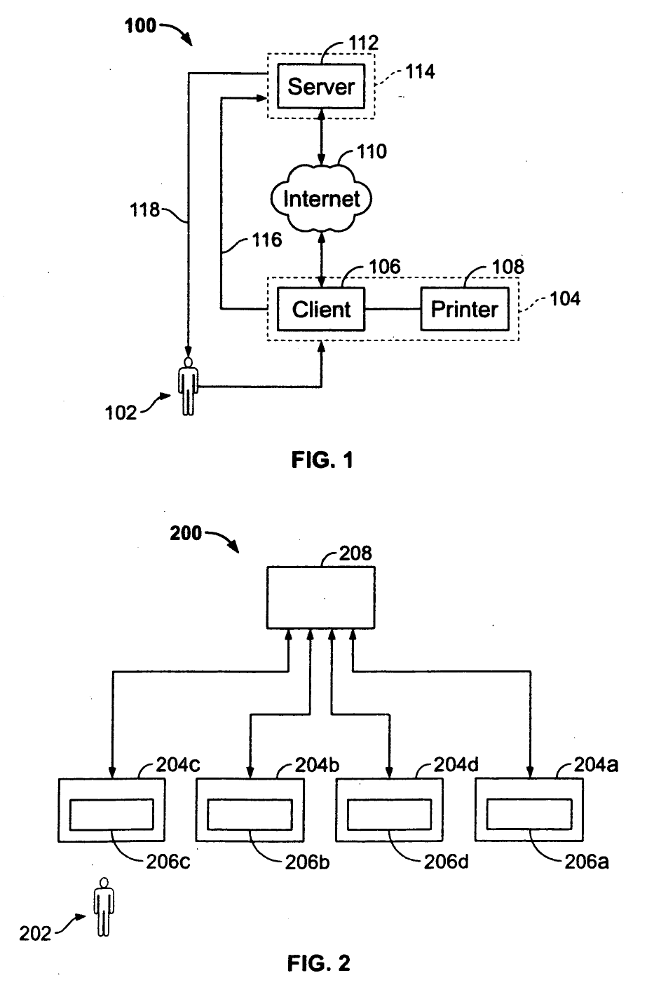

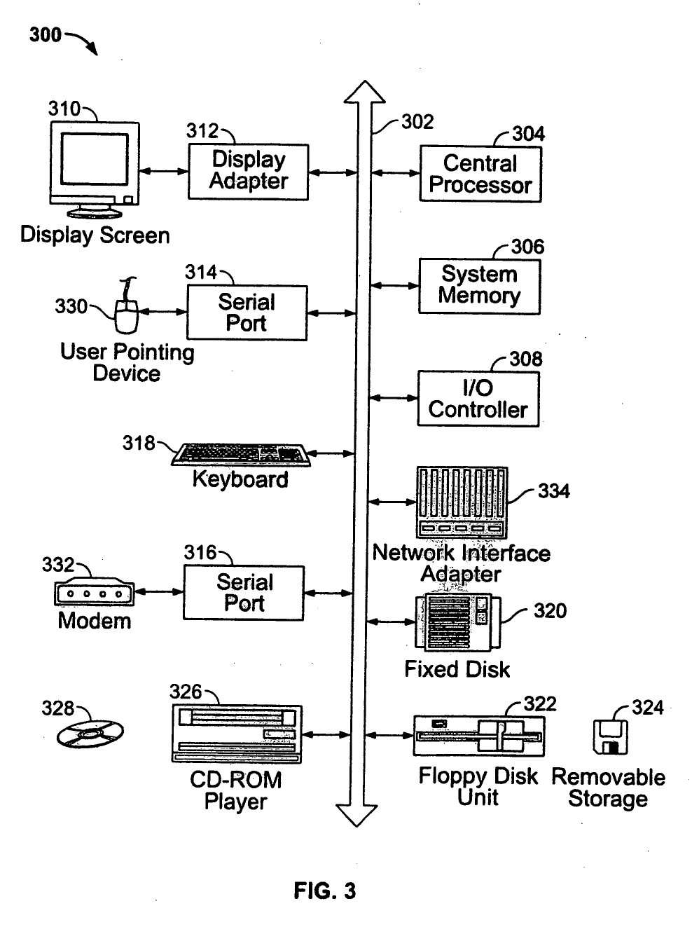

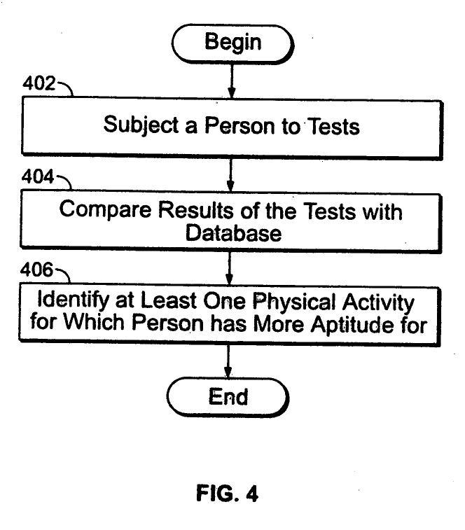

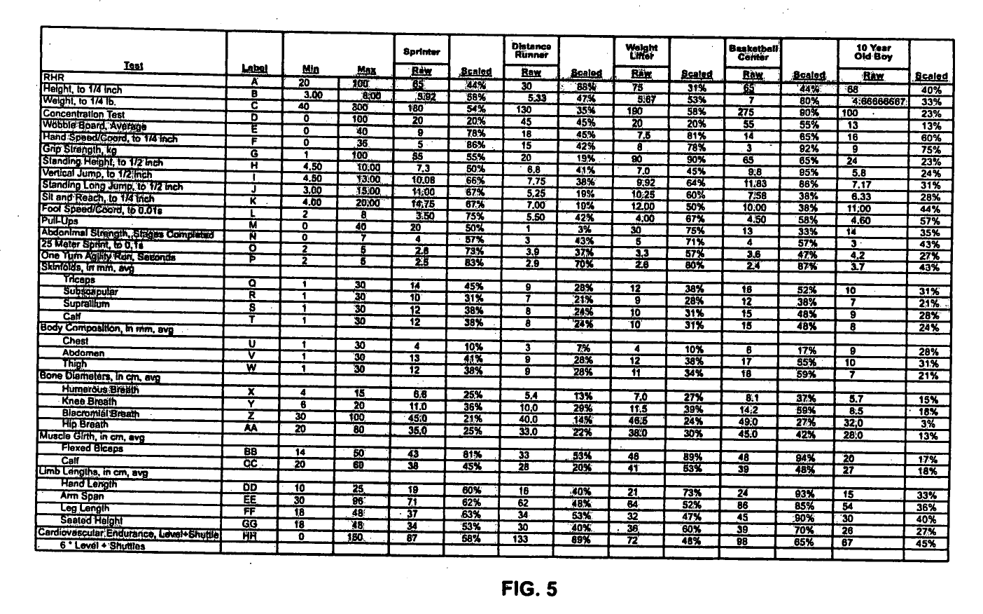

FIG. 1 illustrates a block diagram of a system 100 for testing and evaluating a person's athletic ability in accordance with one embodiment of the present invention. A person 102 is subjected to several tests at a testing location 104. The tests may include but are not limited to physical, physiological, and psychological tests. Examples of the type of tests are described in more details below in FIG. 5. The testing location 104 may also comprise a client 106 coupled to a printer 108. The client 106 may include a computing system and a display, such as a monitor (not shown). The results of the tests of the person 102 are entered on the client 106 and submitted through the internet 110 to a server or computer system 112 located at an analysis location 114. The server 112 is described in more detailed in FIG. 3. The server 112 analyses and compares the data received from the client 106 with a database (not shown) coupled to server 112. The database contains the results of the same tests subjected on a set of athletes from different sports, sports positions, disciplines, and physical activities. The analysis determines which physical activity the person 102 has more aptitude for based on the results of his/her tests at the testing location. The analysis is described in more details below in FIG. 4.

In accordance with one embodiment, the results of the analysis performed by the server 112 may be downloaded at the client 106. The person 102 may be able to view his/her results of the analysis on the client's display monitor or obtain a hardcopy of his/her results from the printer 108.

In accordance with another embodiment of the present invention, the testing location 104 may submit the results of the tests for analysis to the analysis location 114 via other means of transportation, such as mail or fax (116). Those of ordinary skills in the art will recognize that there exists many other ways, electric or non-electric means, of submitting the results of the tests for analysis to the analysis location 114. The results of the analysis may also be sent directly (118) via mail or other means to the person 102 mailing address.

FIG. 2 illustrates a system 200 for evaluating a person's athletic ability in accordance with another embodiment of the present invention. A person 202 may be tested at any of the plurality of testing locations 204a, 204b, 204c, 204d. Each testing location 204a, 204b, 204c, 204d includes respectively a set of tools 206a, 206b, 206c, 206d for testing the person 202. For illustration purposes, the tools 206 may include a stop watch, a scale with body height measurement, a wobble board, a yardstick, a grip, a standing long jump test mat, a pull-up bar, etc. Other equipment may also be needed. The results of the tests of the person 202 may be submitted by the testing facility that tested the person 202 to the analysis center 208 via means described in FIG. 1. The results of the analysis may be sent directly to the person 202 or to any of the location 104.

FIG. 3 depicts a block diagram of a server 112 or computer system 300 suitable for implementing aspects of the present invention. As shown in FIG. 3, computer system 300 includes a bus 302 which interconnects major subsystems such as a central processor 304, a system memory 306 (typically RAM), an input/output (I/O) controller 308, an external device such as a display screen 310 via display adapter 312, serial ports 314 and 316, a keyboard 318, a fixed disk drive 320, a floppy disk drive 322 operative to receive a floppy disk 324, and a CD-ROM player 326 operative to receive a CD-ROM 328. Many other devices can be connected, such as a pointing device 330 (e.g., a mouse) connected via serial port 314 and a modem 332 connected via serial port 316. Modem 332 may provide a direct connection to a remote server via a telephone link or to the Internet via a POP (point of presence). Alternatively, a network interface adapter 334 may be used to interface to a local or wide area network using any network interface system known to those skilled in the art (e.g., Ethernet, xDSL, AppleTalkTM).

Many other devices or subsystems (not shown) may be connected in a similar manner. Also, it is not necessary for all of the devices shown in FIG. 3 to be present to perform or operate the present invention, as discussed below. Furthermore, the devices and subsystems may be interconnected in different ways from that shown in FIG. 3. The operation of a computer system such as that shown in FIG. 3 is readily known in the art and is not discussed in detail in this application, so as not to overcomplicate the present discussion. Code to implement the present invention may be operably disposed in system memory 306 or stored on storage media such as fixed disk 320, floppy disk 324 or CD-ROM 328.

The computer system of FIG. 3 includes a database (not shown) stored in system memory 306 or stored on storage media such as fixed disk 320, floppy disk 324 or CD-ROM 328. The database includes results of the same tests performed on a set of athletes from different fields of sports. For example, the database may include results of physical, physiological, and mental tests of a set of athletes from different sports, sports positions, disciplines, and physical activities such as: a sprinter, distance runner, weightlifter, and a basketball center as illustrated in FIG. 5. The previously cited disciplines are for illustration purposes only.

The computer system 300 receives the test results of a person and compares them with the results of the same tests of a set of athletes from different sports, sports positions, disciplines, and physical activities. The central processor 304 processes the data and correlates at least one sport for which the person has the most aptitude for based on the data in the database. This database may be developed by testing several athletes from the same sport, sports position, discipline, and physical activity or from different sports, sports positions, disciplines, and physical activities. The database may also be updated periodically for accuracy by testing another set of athletes or the same set of athletes.

FIG. 4 is a flow diagram illustrating a method for evaluating a person's athletic ability in accordance with one embodiment of the present invention. At 402, a person is subjected to a set of tests at a testing location. The tests may include physiological tests, physical activity tests, and psychological tests. Those of ordinary skills in the art will recognize that other variables and/or criteria based on the subject may also be taken into consideration in the present analysis.

The physiological tests may include, but are not limited to, a resting heart rate (RHR) test, a height test, a weight test, skinfold tests, a body composition test, bone diameter tests, muscle girth tests, and limb length tests.

The resting heart rate (RHR) test is a test measuring the resting heart rate (RHR) of the subject. The subject is first seated quietly for five minutes. The RHR may be obtained between the fourth and fifth minute. The RHR may be obtained either by using a heart rate monitor attached to the chest strap or using a stop watch to count the number of heart beats in 15 seconds.

The height test is a test measuring the height of the subject to the nearest ⅛ of an inch. The height test may be achieved using a medical scale with a body height determination tool. As the tool is brought in contact with the most superior part of the head, the subject is instructed to take a deep breath and stretch up to their full height while maintaining a flat-footed stance. No shoes or socks should be worn.

The body weight test is a test measuring the weight of the subject to the nearest ¼ of a pound. A calibrated medical scale may be used with the subject standing quietly on the scale.

The skinfold tests are tests measuring the skinfolds of the subject at different sites on the body of the subject. For example, a skinfold may be measured at the following non-limiting locations: triceps, subscapular, suprailium, and calf. A skinfold caliper to measure to the skinfold. A measuring tape may be used to help determine the skinfold location. Those of ordinary skills in the art will recognize that other tools may also be used for the measuring the skinfold of the subject.

The body composition test is a test that measures the fat mass and fat-free mass of the subject's body. The body composition is determined using several of the skinfold measurements and established formulas. The body composition is another indicator of potential ability to participate in a certain sport or activity. Generally the body is considered to be composed of two major components: fat mass or all the adipose in the body and fat-free mass or all other tissues except adipose. Fat-free mass is therefore composed of muscle, bone and all other tissues of the body. Both of these variables can be expressed as a percent of total body weight. Generally, many athletes have a less than normal percent of body fat or % fat. The average body fat for males is about 15%. For the example, athletes' values for % fat for a sprinter, a distance runner, a weightlifter and a basketball center would be respectively 6, 8, 9, and 15%.

The bone diameter tests are tests that measure the bone diameters at different locations on the body of the subject. For example, the bone diameter may be measured at the following non-limiting locations: humerous breath, knee breath, biacromial breath, and hip breath. A small sliding caliper may be used for the bone diameter test. Those of ordinary skills in the art will recognize that other tools may also be used for measuring bone diameters.

The muscle girth tests are tests that measure the girth of the muscle at different locations on the body of the subject. For example, the muscle girth may be measured at the following non-limiting locations: flexed biceps and calf. A measuring tape may be used for the muscle girth test. Those of ordinary skills in the art will recognize that other tools may also be used for the measuring the girth of muscles.

The limb length tests are tests that measure the length of different limbs and body parts on the body of the subject. For example, the limb length may be measured at the following non-limiting locations: hand length, arm span, and leg length. A measuring tape or yardstick may be used for a limb length test. Those of ordinary skills in the art will recognize that other tools may also be used for the measuring the length of the different limbs or body parts.

The physical activity tests may include, but are not limited to, a wobble board test, a hand speed coordination test, a grip strength test, a vertical jump test, a standing long jump test, a sit and reach test, a foot speed coordination test, a pull-up test, an abdominal strength test, a 25-meter sprint test, a one-turn agility run test, and a cardiovascular endurance test.

The wobble board test is a test that measures the balancing capability of the subject by counting the number of times the ends of a wobble board touch the ground during a 30-second period. The subject stands flat-footed on the wobble board with feet on marked places at the ends of the wobble board. The subject is informed to keep the ends of the wobble board from touching the ground while attempting to balance the wobble board.

The hand speed coordination test is a test that measures the hand speed coordination of the subject. A yardstick and a normal height desk or table may be used for the above test. The subject first stands bending over the edge of the table or desk with his/her dominant hand forearm flat on the edge of the desk or table. The subject then places the opposite hand on their dominant hand forearm to assist in keeping the forearm stationary. The subject's little finger side of hand is flat on and just at the edge of the desk or table. The subject's fingers are extended and thumb is pointing up ward and at a 90-degree angle to the fingers. The yardstick is held perpendicular to the floor so that the end is even with the top of the index finger and so that its width is perpendicular to the palm of the hand. The one-inch end is at the bottom of the yardstick. The yardstick is positioned (2-4 inches from the palm) so that when the subject brings the thumb and fingers together it will be caught. The subject is instructed to watch their hand and the end of the yardstick and to catch the yardstick as quick as possible by moving only their fingers and thumb when they see the yardstick move. The yardstick is dropped between 1-5 seconds after the subject is told the yardstick will be dropped. The yardstick is dropped and the number of inches the yardstick drops is recorded at the top of the index finger.

The grip strength test is a test that measures the subject's strength of the grip. A grip or hand dynamometer may be used for this test. In this test, the subject stands looking straightforward. The grip size is adjusted so that the middle finger's (third digit) second phalanx is at approximately a right angle when gripping the dynamometer. The dynamometer is adjusted by slipping off the movable handle and repositioning it. The subject's forearm can be in any elbow position between 90 and 180 degrees (right angle to straight). The subject's wrist should be at its mid position. The subject then squeezes maximally and quickly for approximately three seconds.

The vertical jump test is a test that measures the maximal vertical jump height of the subject. A Vertec slat/vain style may be used for the above test. In this test, the standing reach height is determined by having the subject stand flat-footed and reaches as high as possible with the dominant hand. The maximal vertical jump height is then determined. The maximum vertical jump is calculated by subtracting the standing reach height from maximal vertical jump height.

The standing long jump test is a test that measures the distance of a subject's long jump. A standing long jump test mat may be used. The subject stands with toes just behind a take-off line, swings the arms backwards, bends knees and hips, and then jumps forward as far as possible. The distance from the take-off line to the heel or any other part of the body that touches the floor nearest the take-off line is measured.

The sit and reach test measures the general flexibility of the subject. This test may be performed using a sit and reach box. With no shoes worn, the subject sits with both feet flat against the sit and reach box. The subject performs three forward bobs as if trying to touch the toes. Keeping the knees straight, the subject slowly leans forward as far as possible and pushes the sliding scale of the sit and reach box with the fingertips of both hands as far forward as possible. The subject must keep hands and fingertips one on top of the other with the palms facing down. The fingertips of one hand should never reach beyond the other during the past. This test measures the distance the sliding scale is pushed from the edge of the box to the nearest ¼ inch. The scale on the sit and reach box will be the same one in all tests.

The foot speed coordination test is a test that measures the subject's agility. An agility ladder with 10 slats inside of slats 16 ⅞ in apart, having a total length first to last slat of 14 ft may be used for this test. A stopwatch and a tape measure may also be used. The agility ladder is stretched to its complete length. The start line one yard from the outside edge of the first slat is marked. The finish line one-yard from the outside edge of the last slat is also marked. The total length from start to finish line is 20 ft. The subject stands with one foot just behind the starting line. A staggered foot position is to be utilized at the start. The subject can not use a crouched start, such as in American football or as if starting a 100 meter sprint. The tester stands at the finish line. At the signal of the tester, the subject must cover the length of the ladder by placing first one foot and then the other between each of the slats. For example, the right foot is first placed between the first set of slats and then with the right foot on the ground, the left foot is placed between the first set of slats. Then the right foot and again the left foot is placed between the next set of slats this process is continued until the entire length of a ladder is completed and the subject crosses the finish line. The tester stops the stop watch when the torso of the subject passes the finish line and also records the number of times if any that a foot hits or touches a slat.

The pull-up test measures the number of complete pull-up performed. A pull-up bar may be used for this test. The bar should be high enough so that the arms and legs are fully extended and feet are not touching the floor. An overhand grip (palms facing forward) with hands slightly wider than shoulder width is utilized. The subject is instructed to start the test from an arm fully extended position and that the chin must be placed over the bar for a successful pull-up. The subject is instructed that the arms must be fully extended between each repetition. The subject is instructed that the legs (hips and knees) must remain straight throughout the test. The tester may assist the subject to the initial starting position on the bar.

The abdominal strength test is a test measuring the number of stages successfully completed by the subject. One each of a 2.5-kg (5.5 lb) and a 5 kg (11-lb) weight plates and a gym mat may be used to perform the test. For all stages, the starting position is lying on the back with the feet flat on the floor and a 90-degree angle at the knees with no shoes worn. The feet should be comfortably apart (approximately hip width apart). The subject's feet are not held and the subject is allowed up to 3 attempts for all stages. All movements are to be performed in a smooth and controlled manner.

A stage is considered unsuccessful if: the subject moves either foot partially or totally off the floor, the subject moves the arms from the stage's testing position, the subject throws their arms or head forward in a jerking manner, the subject lifts hips off of the floor, the subject moves the knees from the 90 degree angle, or the subject is unable to complete the testing movement.

In stage one, the subject places the palms of both hands on the same side thighs (right hand on right thigh and left hand on left thigh) with the subject's arms straight. The subject moves forward in a crunching movement until the fingers are touching the patellas (knee caps).

In stage two, the subject places the palms of both hands on the same side thighs with the subject's arms straight. The subject moves forward in a crunching movement until both elbows touch the patellas.

In stage three, the subject's elbows are bent at 90 degrees and the arms are in contact with the abdomen. The subject's hands gripping the opposite elbow. The subject moves forward in a crunching movement until the forearms touch the midthighs.

In stage four, the subject's elbows are bent and the arms are in contact with the chest. The subject's hands grip the opposite shoulder. The subject moves forward in a crunching movement until the elbows touch the midthighs.

In stage five, the subject's arms are crossed behind the head. The subject's hands are gripping the opposite shoulder. The subject moves forward in a crunching movement until the chest touches the thighs.

In stage six, the subject's arms are crossed behind the head with the subject's hands holding a 2.5-kg weight plate. The subject moves forward in a crunching movement until the chest touches the thighs.

In stage seven, the subject's arms are crossed behind the head with the subject's hands holding a 5-kg weight plate. The subject moves forward in a crunching movement until the chest touches the thighs.

The 25-meter sprint test is a test that measures the subject's sprint time over a distance of 25 meters.

The one-turn agility run test is a test that measures the subject's time to perform one turn or change of direction. A stopwatch may be used to perform the test. The subject stands just behind a start line. At the command of the tester the subject sprints 15-meters to a marked line and changes direction 180 degrees and then sprints an additional five meters. The tester stands at a line five meters in front of the line where the subject will change direction and 10 meters from the start line. The tester determines the total time it takes the subject to sprint from five meters in front of the line at which the subject changes direction, time to change direction and sprint five meters back to the tester.

The cardiovascular endurance test is a test that measures the level and the shuttle number completed using a 20-meter shuttle run test CD. There may be a total of 22 levels and 10 shuttles. The test involves the subject jogging and then running back and forth between two lines 20 meters apart at successively faster paces. The test pace is determined by beeping sounds. The time between each beep decreases and so running speed correspondingly increases. The subject is allowed to warm up by running and stretching prior to the test. Starting the test at level 1, the subject must always place one foot on or behind the lines at the sound of each beep. If a subject fails to reach a line at the sound of a beep they are warned the test will be stopped. When a subject fails to reach 2 successive lines at a beep the test is terminated. After completing the test, the subject cools down by walking slowly.

In accordance with one embodiment of the present invention, the psychological tests may include personal preferences and interests tests. The psychological tests may be designed to measure the concentration level, confidence level, control skills, commitment level, leadership skills, adaptability skills, and socialization skills by subjecting the person to a questionnaire. The questionnaire may include several redundant questions for accuracy purposes. Such psychological tests may be conducted independently from the physical activity and physiological tests. Accordingly, the analysis based on the results of the physical and physiological tests may also be conducted independently from the results of the psychological tests. The psychological testing may be optional and additionally performed to further extensively evaluate the person's physical ability. For example, a subject who has demonstrated a physical aptitude of a swimmer may be afraid of the water.

In accordance with another embodiment of the present invention, the above tests may be conducted in many other ways. For example, the subject may be subjected to fewer tests and/or shorter tests that would be still representative of the aptitude of the subject. However, the accuracy of the analysis may increase with the number of tests and length of each test.

Those of ordinary skill in the art will appreciate that the above-specified tests are not intended to be limiting and that other tests can be used without departing from the inventive concepts herein disclosed. The accuracy of the present invention increases with the number of different tests and the repetition of a single test. However, for economic and practical reasons, a limited number of tests has been selected. In accordance with one embodiment of the present invention, such limited number of tests should be conducted within, for example, 90-120 minutes.

After the person is subjected to the tests at 402, the tests results and/or the other variables are compared with a database at 404. The database includes the results of the same or similar tests described above subjected on a set of athletes from different sports, sports positions, discipline or physical activities. For example, the physical activities may include sports such as football, basketball, track and field. In accordance with one embodiment, the database may further include the results of the same or similar tests applied to the athletes in specific positions within the same sport. For example, the database may store the results of the tests subjected on a basketball center, or a basketball guard.

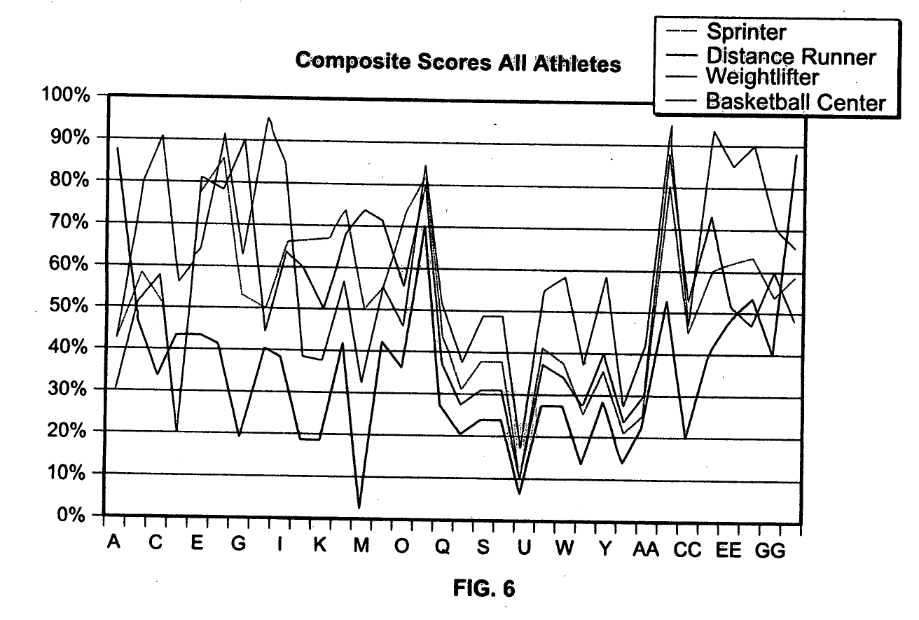

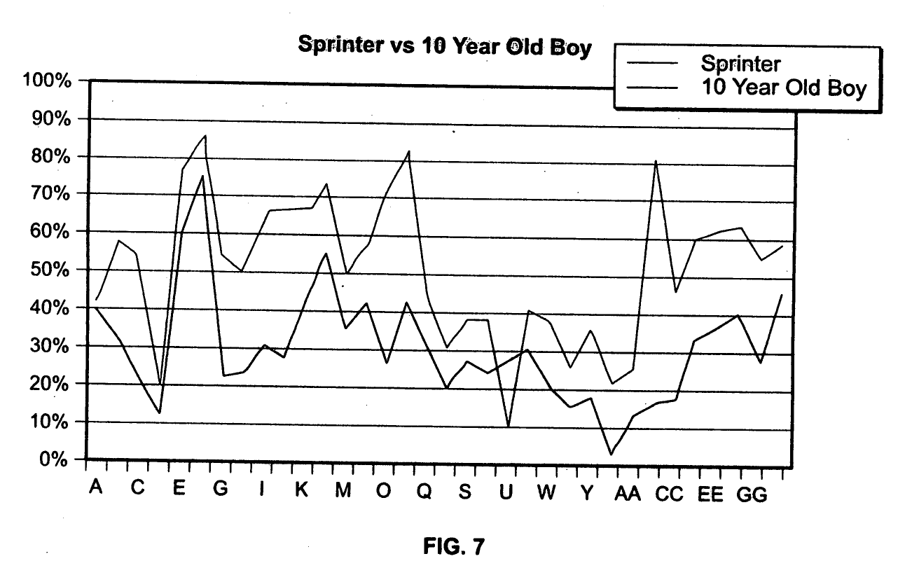

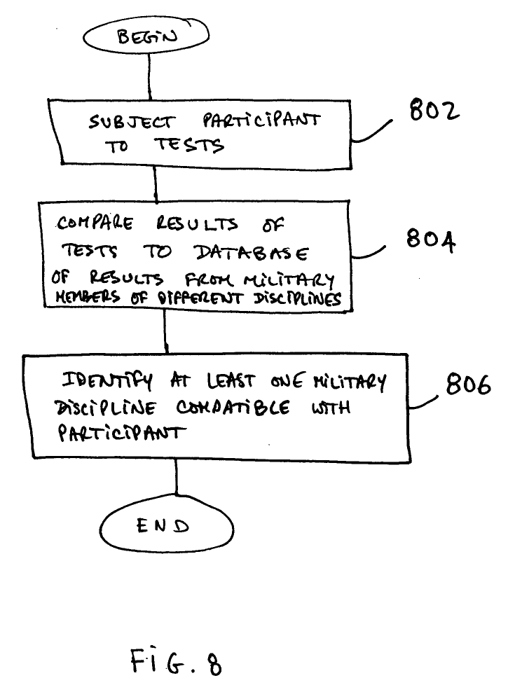

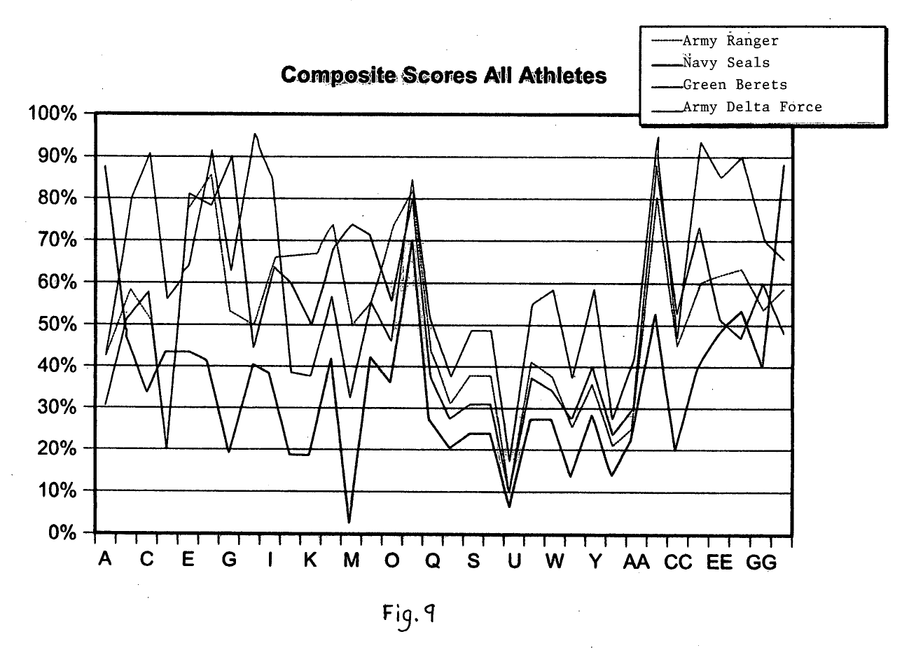

After the subject's tests results and other variables based on the subject are compared with the database at 404, a specific physical activity with tests results in the database closest to the subject's tests results is identified at 406. In accordance with one embodiment of the present invention, the data are displayed in a plot on a graph or other visual display along with the results of the same or similar tests on the set of athletes from different sports, sports positions, discipline, and physical activities. The comparison may also include the other variables based on the subject. For example, other variables may include the siblings' heights of the subject. The data (the subject's test results and the other variables) during the comparison step in 404 are scaled for comparison purposes. The identification may be accomplished by selecting the plot of the average results of a set of athletes from a specific physical activity that most closely matches the plot of the subject's data. This is illustrated in more detailed in FIGS. 6 and 7.

In accordance with another embodiment, the subject may also receive a report showing a graph or other visual display explaining which sports, sports positions, discipline, or physical activities, the subject has more aptitude for based on the tests results and the above identification. The report may also show which sports, sports positions, discipline, or physical activities for which the subject has less aptitude. For example, the report may list three sports with which the subject may be more compatible, and three sports with which the subject may be less compatible.

FIG. 5 is a table illustrating an example of test results on a set of athletes in four different physical activities with a subject's test results in accordance with one embodiment of the present invention. The following tests are listed in the first column: RHR, Height, Weight, Concentration Test, Wobble Board Test, Hand Speed-Coordination Test, Grip Strength Test, Standing Height Test, Vertical Jump Test, Standing Long Jump Test, Sit and Reach Test, Foot Speed-Coordination Test, Pull-up Test, Abdominal Strength Test, 25-meter Sprint Test, One Turn Agility Run Test, Skinfold Tests, Body Composition Test, Bone Diameters Tests, Muscle Girth Tests, Limb Lengths Tests, and Cardiovascular Endurance Test.

The second column indicates the horizontal labels on FIGS. 6 and 7.

The third and fourth columns illustrate respectively the minimum and maximum possible measurement for each test. For example, the minimum RHR may be 20 heartbeats within one minute while the maximum RHR may be 100 heartbeats within one minute.

The fifth and sixth columns illustrate respectively the median or average raw data obtained from the test and the median or average scaled results of a set of sprinters. For example, the average sprinter may have a RHR of 65 heartbeats within one minute. The scaled results using the minimum and maximum in columns two and three is 44%.

The seventh and eighth columns illustrate respectively the median or average raw data obtained from the test and the median or average scaled results of a set of distance runners. The ninth and tenth columns illustrate respectively the median or average raw data obtained from the test and the median or average scaled results of a set of weightlifters. The eleventh and twelfth columns illustrate respectively the median or average raw data obtained from the test and the median or average scaled results of a set of basketball centers.

The thirteenth and fourteenth columns illustrate respectively the raw data obtained from the test and the scaled results of a subject such as a 10-year-old boy.

FIG. 6 is a graph illustrating an example of the median or average tests results of a set of sprinters, long distance runners, weightlifters, basketball centers from FIG. 5, in accordance with one embodiment of the present invention. The scaled results of the different athletes from the sixth, eighth, tenth, and twelfth columns are plotted on the graph. The vertical axis represents the scaled results in terms of percentage. The horizontal axis represents each test as described above with each corresponding label from the second column of FIG. 5.

FIG. 7 is a graph illustrating an example of a correlation between the results of a test subject and a set of sprinters in accordance with one embodiment of the present invention. The scaled results of the subject from column 13 are plotted on the graph along with the scaled results of an identified set of athletes with close or similar test results. The vertical axis represents the scaled results in terms of percentage. The horizontal axis represents each test as described above with each corresponding label from the second column of FIG. 5. In the present example, the 10-year-old boy has more aptitude to become a sprinter based on the results of his test than to become a long distance runner, weightlifter, or basketball center.

In accordance with another embodiment, the above illustrated method may be applied to evaluate a participant's physical ability to determine the most compatible fields in a military setting. For instance, various elite military units, such as the Green Berets, Seals, Rangers and elite pilots, have several physical, physiological, anatomical and psychological characteristics in common. All of these elite military units demand the highest physical and psychological conditions and these characteristics can be tested. However, there are also differences between the elite military units: Seal divers perform many of their operations in water while pilots perform many of their operations against increased gravitational forces. The physical, psychological and anatomical characteristics necessary to perform operations in these unique environments may also be tested using the above method with different or modified sets of tests.

Likewise, the traditional soldier, sailor, marine, air, and other military personnel also have some operational duties in common. However, like the elite units, different units within each branch of the military have physical and psychological tasks unique to their specific unit. For example, individuals within transportation units have tasks such as loading and unloading trucks, loading and unloading airplanes as well as other tasks related to movement of supplies and equipment.

In accordance with another embodiment of the present invention, the physical and psychological tests used to determine capability to perform elite military tasks as well as traditional combat military tasks may include the following: cardiovascular endurance, muscular strength of various muscle groups (grip strength for tasks such as rope and rock climbing), short sprint ability, vertical jump ability, standing long jump ability, agility, and agility endurance (long duration agility tests), load carrying (back pack, weapon) sprint ability, shuttle run or change of direction ability, load carrying endurance ability, load carrying uphill ability, and load carrying climbing ability. The difference between elite military units as well as traditional military branches will be apparent based on their operational specialties. Other physical characteristics such as vision, night vision, contrast vision, and ability to perform a monotonous task, such as guard duty, may also be necessary for various operational conditions and are testable characteristics. Those of ordinary skills in the art will recognize that there may be other types of tests used for the military application.

Information concerning physiological characteristics necessary for success and compatibility in some military tasks is available. For example, loaded march (carrying a pack) performance appears to rely on a combination of strength and endurance. With respect to females in the military, their expanding role also offers some potentially testable criteria for traditional military related tasks. For example, stronger women perform better with a maximal box lift and endurance box lift. A box lift task is the task of loading and unloading boxes to a truck or other vehicle.

Additionally, some tests can distinguish which military recruits or participants will respond favorably to a specific type of training such as load carriage ability. For example, individuals who respond best to circuit type weight training to increase load carrying ability have significantly lower ratios of performance in a shuttle run endurance test to isometric pull strength and shuttle run endurance performance to lower back extension strength than those who showed a small increase in load carrying ability. Thus a testing battery of physiological and psychological parameters will not only allow prediction of which individuals are best suited for particular units or military tasks, but also indicate what type of training individuals should undertake to improve performance of various military tasks.

Psychological characteristics also differentiate elite military units from individuals in traditional military units, the normal population and from each other. Elite unit members display some common psychological characteristics, such as aggressiveness, ability to perform under physical and psychological stressors, and ability to make decisions based upon minimal and uncertain information. Intelligence (IQ) and ability to learn and master quickly foreign languages are also psychological characteristics that some elite units need to possess. While psychological tests on the more traditional military units may also indicate which individuals are most adept at performing various combat and noncombat tasks.

Anatomical characteristics, such as height, weight, leg length, torso length and arm length may also distinguish between some elite military units as well as individuals who will be most adept at performing certain combat and noncombat tasks. For example, load carrying ability may be related to torso and leg length relationships.

Sports play is an important role in each branch of our military and generally throughout all levels of the service. Student athletes at the Air Force, Army, and Navy military academies have a minimum of 15 intercollegiate athletic teams and a variety of club sports to choose from. Both the Army and Air Force have a World Class Athlete Program which enables these services to identify and train future Olympians. The scientifically based scoring system, tests, and measurements used in the method of presently claimed invention can be modified to test anatomical, psychological, physiological and other characteristics necessary to the development of an efficient and effective military. One of the primary military applications for Sports Potential's service is comparing the physical, physiological, anatomical and psychological traits body types of soldiers in specialized elite units throughout the services such as the Navy SEALS, Army Delta Force, Submariners, Green Berets, Army Rangers, and Naval Aviators, etc., against the sports they play to determine if correlations exist between particular sports and those members of specialized specific special forces units. For example, the body type of a Navy Seal may correspond to that of a triathlete while an Army Delta team member may have a body type similar to a wrestler or a football linebacker. The scientific and numeric data obtained through the assessment will enable commanding officers in each branch of the military to identify sports which can become feeders for various special forces units. Alternatively, different units within each branch of the military have physical and psychological tasks unique to their specific unit as mentioned previously. Sports Potential's assessment tools and measurements can be modified to test these troops. The results can be used to reallocate resources, improve overall troop performance, efficiency and effectiveness.

The military may use the presently claimed method as part of the recruitment process. For example, the tests can be given to determine strengths and weaknesses such as endurance and power as well as to identify the most appropriate military role/placement. Similarly, the same can be done for officers and individuals who attend Officer Candidate School. The method described above can be used to facilitate the development of an ideal candidate profile. The military services can then refer to these results as they plan for and engage in the annual recruitment process.

FIG. 8 is a flow chart illustrating another embodiment from the present invention. At 802, a participant is subjected to several tests. At 804, the results of the tests on the participant are compared with a database. The database contains the results of the same tests subjected to a set of elite military members from different disciplines. At 806, at least one specific discipline compatible with the participant is identified based on the comparison and correlation analysis.

By integrating the testing services described above into each of the three areas outlined above, the military can use the results to improve talent identification and troop resource allocation which is becoming increasingly important with the demands placed on the Army, Navy, Air Force, and Marines by the war on terror in addition to more traditional commitments.

Reference data for the selected tests would be obtained by testing the best squadrons or group of individuals in each of the elite and other military units. While reference data for the more traditional military units could be obtained by testing recruits prior to and after their initial military training. Reference data for characteristics necessary to perform the specialized tasks performed by each of the elite units would be obtained by testing the individuals within each of the elite military units whom perform a certain task the best. For example, the data for Seals who perform best in a five mile underwater swim carrying full gear could be utilized to develop a test profile for this task. While the data for individuals who perform best in a 10 mile march in full gear could be utilized to develop a test profile for this task. Reference data for other military groups could be obtained from groups demonstrating the best performance in such things as military maneuvers or contests, such as, the “top gun” competitions held for pilots. Reference data to determine which characteristics best predict ability to perform tasks such as load carriage ability or truck loading ability could be obtained by actually testing the task (i.e. box loading ability) as well as the entire battery of physical, physiological and psychological tests. Statistical comparisons between results on the physical, physiological and psychological test battery could then be made to all of the tasks performed by elite or more traditional military units.

While embodiments and applications of this invention have been shown and described, it would be apparent to those skilled in the art having the benefit of this disclosure that many more modifications than mentioned above are possible without departing from the inventive concepts herein. The invention, therefore, is not to be restricted except in the spirit of the appended claims.

What is claimed is:

1. A method for evaluating a participant:
subjecting the participant to a plurality of tests;
comparing the results of said plurality of tests with a database, said database including the results of said plurality of tests subjected to a set of elite military members from different disciplines; and
identifying at least one specific discipline compatible with the participant based on said comparing.

2. The method of claim 1 further comprising:
sorting into a list said identified specific disciplines from most compatible to least compatible associated with a corresponding match score.

3. The method of claim 1 wherein said plurality of tests further comprises a plurality of physical activity tests, a plurality of physiological tests, and a plurality of psychological tests.

4. The method of claim 3 wherein said plurality of physical activity tests comprises:
cardiovascular endurance, muscular strength of various muscle groups, short sprint ability, vertical jump ability, standing long jump ability, agility, and agility endurance, load carrying sprint ability, shuttle run, change of direction ability, load carrying endurance ability, load carrying uphill ability, and load carrying climbing ability.

5. The method claim 3 wherein said plurality of physiological tests further comprises a resting heart rate test, a height test, a weight test, a standing height test, skinfold tests, a body composition test, bone diameter tests, muscle girth tests, limb and length tests.

6. The method of claim 3 wherein said plurality of psychological tests further comprises a concentration test and a questionnaire test.

7. The method of claim 1 wherein said database is periodically updated with a new set of elite military members from different disciplines.

8. The method of claim 1 wherein said discipline includes a unit of a military specialized force.

9. The method of claim 1 wherein said discipline includes a unit of the military.

10. The method of claim 2 further comprising:
displaying at least one graph illustrating the results of a set of elite military members from different disciplines with respect to the results of the participant.
